(19) United States
(12) Patent Application Publication
(10) Pub. No.: US 2022/0225954 A1
(43) Pub. Date: Jul. 21, 2022

(54) VIRTUAL DETERMINATION OF BASELINE IMAGES IN CT PERFUSION MEASUREMENT

(71) Applicant: Siemens Healthcare GmbH, Erlangen (DE)

(72) Inventors: Markus JUERGENS, Adelsdorf (DE); Bernhard SCHMIDT, Fuerth (DE)

(73) Assignee: Siemens Healthcare GmbH, Erlangen (DE)

(21) Appl. No.: 17/575,966

(22) Filed: Jan. 14, 2022

(30) Foreign Application Priority Data
Jan. 21, 2021 (DE) 102021200544.6

(51) Int. Cl. A61B 6/00; A61B 6/03

(52) U.S. Cl. CPC A61B 6/5205; A61B 6/032; A61B 6/507; A61B 6/503; A61B 6/482; A61B 6/481; A61B 6/501; A61B 6/4241

(57) ABSTRACT

A CT perfusion data determination method is described. In the CT perfusion data determination method, raw X-ray data affected by contrast agent and generated by way of a spectral or multi-energy CT image acquisition method is recorded from an examination region, a plurality of images having been acquired from the examination region at successive time instants. Based on the recorded raw X-ray data, a virtual baseline image is determined by calculating virtual native image data through the application of a material decomposition. Finally a temporal course of a contrast agent concentration in the examination region is determined based upon the raw X-ray data affected by contrast agent and the virtual baseline image. A CT perfusion measurement method is also described. Furthermore, a CT perfusion data determination facility is described. Moreover, a computed tomography system is described.

### PRIORITY STATEMENT

The present application hereby claims priority under 35 U.S.C. § 119 to German patent application number DE 102021200544.6 filed Jan. 21, 2021, the entire contents of which are hereby incorporated herein by reference.

### FIELD

Example embodiments of the invention generally relate to a CT perfusion data determination method. In the CT perfusion data determination method, raw X-ray data affected by contrast agent and generated by way of a spectral or multi-energy CT image acquisition method is recorded from an examination region, wherein a plurality of images have been acquired from the examination region at successive time instants. Example embodiments of the invention also relate to a CT perfusion measurement method; a CT perfusion data determination facility; and a computed tomography system.

### BACKGROUND

For the clarification of medical questions, X-ray imaging devices, such as for example a computed tomography device, CT device for short, are being used ever more frequently.

One possibility for determining the perfusion of organs, for example the brain or the liver, and thus enabling the viability of the organs to be investigated in more detail consists in carrying out a perfusion measurement. Such a perfusion measurement can be carried out for example with a CT device.

During the CT perfusion measurement, a multiplicity of CT acquisitions are carried out in temporal succession in order to record the flow behavior of a contrast agent through an examination region. Such an examination region can comprise for example the brain, the liver or the heart. For example, during such a perfusion scan, 50 images are acquired in temporal succession from an examination region while the contrast agent flows through this examination region. In order to be able to determine the concentration of contrast agent in the examination region in a spatially resolved manner, it is necessary to know not only the contrast of the contrast agent but also the original contrast in the examination region or at all points of the examination region which would be measured without contrast agent in the examination region. For this purpose, native images without contrast agent are acquired. Typically, five native images are acquired during a so-called baseline scan or baseline acquisitions, also referred to as precontrast images. The quantity of contrast agent introduced is then proportionate to the difference in the attenuation values of the contrast agent images and the images acquired without contrast agent, in other words the native images. Native images are therefore absolutely necessary in order to be able to evaluate a perfusion measurement.

To be able to carry out baseline acquisitions, these must conventionally be carried out shortly before the arrival of the contrast agent bolus. This is however at the expense of a higher X-ray dose, as a part of the X-ray dose must be used for the additional acquisitions before the arrival of the contrast agent bolus in the examination region. Moreover, the workflow is made more complicated thereby. For example, a test bolus is required with which the time between the injection and the arrival of the bolus is determined.

Conventionally, a baseline volume is calculated via averaging over the first volume of a CT perfusion acquisition. So that at least the image during measurement of the first volume is contrast-free, different techniques are conventionally used. For example a fixed time span, also referred to as “delay”, can be defined between the contrast agent injection and the start of the first image acquisition. However, this procedure requires a longer overall acquisition time in order to take account of the variability of the human physiology and to ensure that the baseline acquisition takes place without contrast agent. It is also possible to use a test bolus in order to determine the time required for a contrast agent to arrive in the examination region for a specific person. However, this makes the overall workflow more complicated and increases the time required. Alternatively, a separate acquisition is conventionally carried out before the start of the perfusion acquisition proper and only thereafter is a perfusion acquisition started with a bolus triggering. In this variant, too, more time and an increased X-ray dose are required in order to carry out the separate CT acquisition of the examination region.

### SUMMARY

At least one embodiment of the present invention is directed to a CT perfusion measurement which can be carried out with reduced effort and preferably in a shorter time and with a lower X-ray dose than is conventionally the case.

Embodiments of the invention are directed to a CT perfusion data determination method, a perfusion measurement method, a CT perfusion data determination facility, and a computed tomography system.

In the CT perfusion data determination method according to an embodiment of the invention, raw X-ray data affected by contrast agent is firstly recorded from an examination region. The raw X-ray data affected by contrast agent has been generated by way of a spectral or multi-energy CT image acquisition method. Raw X-ray data affected by contrast agent is to be understood to mean raw X-ray data that has been acquired in the presence of an X-ray contrast agent. Here, X-ray attenuation values or CT attenuation values are changed at least with suitable X-ray energies in comparison to an image acquisition without contrast agent. During the raw data acquisition, a plurality of images have been acquired from the examination region at successive time instants. Based upon the recorded raw X-ray data which has been recorded for several spectral ranges of the X-ray energy, a virtual baseline image is determined by calculating virtual native image data through the application of a material decomposition either directly to the recorded raw X-ray data in the raw data space or through the application of a material decomposition to reconstructed image data after a reconstruction of the image data based upon the raw X-ray data in the image data space.

In the CT perfusion measurement method according to an embodiment of the invention, firstly a spectral or multi-energy CT image acquisition method is carried out, wherein raw X-ray image data affected by contrast agent is generated from an examination region and a plurality of images are acquired from the examination region at successive time instants. Furthermore, the CT perfusion data determination method according to an embodiment of the invention is carried out based upon the acquired raw X-ray data affected by contrast agent. The CT perfusion measurement method according to an embodiment of the invention shares the advantages of the CT perfusion data determination method according to an embodiment of the invention.

The CT perfusion data determination facility according to an embodiment of the invention comprises a data recording unit. The data recording unit is configured to record raw X-ray data affected by contrast agent, which has been generated by way of a spectral or multi-energy CT image acquisition method, from an examination region. Here, the recording of the raw X-ray data comprised an acquisition of a plurality of raw X-ray datasets, wherein therefore a plurality of images were acquired from the examination region at successive time instants. An image determination unit for determining a virtual baseline image by calculating virtual native image data by way of a material decomposition based upon the recorded raw X-ray data is also part of the CT perfusion data determination facility according to an embodiment of the invention. The CT perfusion data determination facility according to an embodiment of the invention also comprises a concentration determination unit for determining a temporal course of a contrast agent concentration in the examination region based upon the raw X-ray data affected by contrast agent and the virtual baseline image. The CT perfusion data determination facility can further comprise an image reconstruction unit, which can be part of the concentration determination unit or can be disposed upstream thereof and which is configured to reconstruct image datasets based upon the recorded raw X-ray datasets which form the basis for a perfusion measurement or based upon which the contrast agent concentration in the examination region is determined. The CT perfusion data determination facility according to an embodiment of the invention shares the advantages of the CT perfusion data determination method according to an embodiment of the invention.

The computed tomography system according to an embodiment of the invention has an X-ray emission unit, a detector unit for recording spectral or multi-energy raw data and the CT perfusion data determination facility according to an embodiment of the invention. The computed tomography system according to an embodiment of the invention shares the advantages of the CT perfusion data determination facility according to an embodiment of the invention.

An implementation largely in software has the advantage that even control facilities of computed tomography systems already in use can be easily upgraded by a software update in order to work in the manner according to an embodiment of the invention. In this respect, an embodiment is directed to a corresponding computer program product with a computer program which is loadable directly into a computed tomography system or a storage facility of a control facility of a computed tomography system and comprises program sections in order to carry out all steps of the CT perfusion data determination method according to an embodiment of the invention and/or the CT perfusion measurement method according to an embodiment of the invention when the computer program is executed in the control facility of the computed tomography system.

An embodiment of the invention is directed to a CT perfusion data determination method, comprising:

recording raw X-ray data affected by contrast agent and generated by way of spectral or multi-energy CT image acquisition, wherein a plurality of images have been acquired from an examination region at successive time instants;

determining a virtual baseline image by calculating virtual native image data by way of a material decomposition based upon the raw X-ray data recorded; and

determining a temporal course of a contrast agent concentration in the examination region based upon the raw X-ray data affected by contrast agent and the virtual baseline image.

An embodiment of the invention is directed to a CT perfusion measurement method, comprising:

carrying out the spectral or multi-energy CT image acquisition, wherein raw X-ray image data affected by contrast agent is generated from an examination region and the plurality of images are acquired from the examination region at successive time instants; and

carrying out the CT perfusion data determination method of an embodiment based upon the raw X-ray data affected by contrast agent recorded.

An embodiment of the invention is directed to a CT perfusion data determination facility, comprising:

a data acquisition unit to record raw X-ray data affected by contrast agent and generated by way of spectral or multi-energy CT image acquisition, wherein a plurality of images have been acquired from the examination region at successive time instants;

an image determination unit to determine a virtual baseline image by calculating virtual native image data by way of a material decomposition based upon the raw X-ray data recorded; and

a concentration determination unit to determine a temporal course of a contrast agent concentration in the examination region based upon the raw X-ray data affected by contrast agent and the virtual baseline image.

An embodiment of the invention is directed to a computed tomography system, comprising:
an X-ray emission unit;
a detector unit to record spectral or multi-energy raw data; and
the CT perfusion data determination facility of an embodiment.

An embodiment of the invention is directed to a non-transitory computer program product storing a computer program, directly loadable into a storage facility of a computed tomography system, including program sections to carry out the method of an embodiment when the computer program is executed in the computed tomography system.

An embodiment of the invention is directed to a non-transitory computer-readable medium storing program sections, readable in and executable by a computer unit, to carry out the method of an embodiment when the program sections are executed by the computer unit.

### BRIEF DESCRIPTION OF THE DRAWINGS

The invention is described and explained in more detail below based upon the example embodiments shown in the figures, in which:

FIG. 1 shows a flow diagram illustrating a CT perfusion data determination method according to an example embodiment of the invention,

FIG. 2 shows a flow diagram illustrating a CT perfusion measurement method according to an example embodiment of the invention,

FIG. 3 shows a schematic representation of a CT perfusion data determination facility according to an example embodiment of the invention,

FIG. 4 shows a schematic representation of a computed tomography system according to an example embodiment of the invention.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

In the CT perfusion data determination method according to an embodiment of the invention, raw X-ray data affected by contrast agent is firstly recorded from an examination region. The raw X-ray data affected by contrast agent has been generated by way of a spectral or multi-energy CT image acquisition method. Raw X-ray data affected by contrast agent is to be understood to mean raw X-ray data that has been acquired in the presence of an X-ray contrast agent. Here, X-ray attenuation values or CT attenuation values are changed at least with suitable X-ray energies in comparison to an image acquisition without contrast agent. During the raw data acquisition, a plurality of images have been acquired from the examination region at successive time instants. Based upon the recorded raw X-ray data which has been recorded for several spectral ranges of the X-ray energy, a virtual baseline image is determined by calculating virtual native image data through the application of a material decomposition either directly to the recorded raw X-ray data in the raw data space or through the application of a material decomposition to reconstructed image data after a reconstruction of the image data based upon the raw X-ray data in the image data space.

For example, during the use of a contrast agent, preferably iodine, not only the X-ray edge, in other words the K-edge of the contrast agent, but generally the different behavior of the contrast agent and water in response to a change in the X-ray spectrum is considered. If there are two images for different parts of the X-ray spectrum, for example in a spectral range of 20 to 50 keV and 100 to 150 keV, then the proportion of water and contrast agent can be calculated by solving a linear 2×2 system, the base material decomposition. The proportion of water is then approximately the virtual native image or virtual baseline image, since fat and soft tissue behave in a similar way to water in terms of the attenuation of X-ray radiation and in this regard differ significantly from a contrast agent or from the attenuation properties of a contrast agent, particularly iodine.

Alternatively, the material proportions can be combined to form mono-energy image data. Here, the material proportions of the X-ray attenuation in either the raw data space or the image data space are combined in such a way that image data with a predetermined high X-ray energy is generated. The virtual image data is then calculated for a high predetermined X-ray energy above the X-ray edge. In other words, the X-ray energy is selected in such a way that a native image is produced, in other words without consideration of the properties of the contrast agent for the selected energy.

Based upon the raw X-ray data affected by contrast agent, which has been recorded at different or successive times, and of the determined virtual baseline image, finally a temporal course of a contrast agent concentration in the examination region is determined. To this end, for example, an X-ray contrast of an image affected by contrast agent can be determined by subtracting the contrast values or X-ray attenuation values of the virtual baseline image from the X-ray attenuation values of the respective image affected by contrast agent, so that in the respective image a spatially dependent X-ray attenuation value reproduces or represents a spatially dependent contrast agent concentration and thus a spatially dependent and time-dependent occurrence of an X-ray contrast agent concentration in the examination region can be determined by way of the images recorded in temporal succession.

Raw X-ray data affected by contrast agent from the examination region is therefore processed, the raw X-ray data having been generated by way of a spectral or multi-energy CT image acquisition method. A spectral CT image acquisition method is to be understood to mean a CT image acquisition method in which an X-ray spectrum is recorded in a resolved manner after at least two X-ray energy intervals. Such a spectrally resolved detection of X-ray radiation can take place for example by way of what is known as a photon-counting X-ray detector. A multi-energy CT image acquisition method involves a separate detection of X-ray radiation with at least two different spectra, for example by way of two separate detectors, which each record a different spectral range of the X-ray radiation transmitted through the examination region during CT imaging. An alternative multi-energy CT image acquisition method involves a separate detection of X-ray radiation with at least two different spectra, which can be generated for example by a periodic change in the voltage of the X-ray source (known as kV switching). Virtual native image data is generated based upon the recorded raw X-ray data. As already mentioned, the virtual native image data is obtained with the aid of a material decomposition. Methods for obtaining image data by way of a material decomposition are described for example in U.S. Pat. No. 7,778,454 B2. McCoullough et al, “Principles and Applications of Multi-energy CT”, Report of AAPM Task Group 291, also discloses the calculation of virtual image data based upon dual-energy and multi-energy CT acquisitions.

An advantage of the approach according to an embodiment of the invention consists in that no separate image acquisition is required for the baseline image, but instead this baseline image can be additionally generated almost as a by-product based upon the images produced during perfusion measurement without any additional time outlay. Advantageously, an acquisition time of a perfusion measurement can be reduced. Furthermore, the approach according to an embodiment of the invention enables a perfusion measurement to be triggered by observing the contrast agent concentration in the patient's body remotely from the actual examination region in the patient's body. In conventional perfusion measurements, such a perfusion measurement must however be started at a very early stage, as after the triggering signal it is still necessary to generate corresponding separate native images, so that a triggering based upon a contrast agent concentration occurring in the patient's body or detected there would come too late. Expressed more generally, the scheduling of the perfusion measurement or the temporal coordination of the contrast agent bolus and the perfusion measurement proper in the examination region can be carried out significantly more precisely.

In the CT perfusion measurement method according to an embodiment of the invention, firstly a spectral or multi-energy CT image acquisition method is carried out, wherein raw X-ray image data affected by contrast agent is generated from an examination region and a plurality of images are acquired from the examination region at successive time instants. Furthermore, the CT perfusion data determination method according to an embodiment of the invention is carried out based upon the acquired raw X-ray data affected by contrast agent. The CT perfusion measurement method according to an embodiment of the invention shares the advantages of the CT perfusion data determination method according to an embodiment of the invention.

The CT perfusion data determination facility according to an embodiment of the invention comprises a data recording unit. The data recording unit is configured to record raw X-ray data affected by contrast agent, which has been generated by way of a spectral or multi-energy CT image acquisition method, from an examination region. Here, the recording of the raw X-ray data comprised an acquisition of a plurality of raw X-ray datasets, wherein therefore a plurality of images were acquired from the examination region at successive time instants. An image determination unit for determining a virtual baseline image by calculating virtual native image data by way of a material decomposition based upon the recorded raw X-ray data is also part of the CT perfusion data determination facility according to an embodiment of the invention. The CT perfusion data determination facility according to an embodiment of the invention also comprises a concentration determination unit for determining a temporal course of a contrast agent concentration in the examination region based upon the raw X-ray data affected by contrast agent and the virtual baseline image. The CT perfusion data determination facility can further comprise an image reconstruction unit, which can be part of the concentration determination unit or can be disposed upstream thereof and which is configured to reconstruct image datasets based upon the recorded raw X-ray datasets which form the basis for a perfusion measurement or based upon which the contrast agent concentration in the examination region is determined. The CT perfusion data determination facility according to an embodiment of the invention shares the advantages of the CT perfusion data determination method according to an embodiment of the invention.

The computed tomography system according to an embodiment of the invention has an X-ray emission unit, a detector unit for recording spectral or multi-energy raw data and the CT perfusion data determination facility according to an embodiment of the invention. The computed tomography system according to an embodiment of the invention shares the advantages of the CT perfusion data determination facility according to an embodiment of the invention.

The essential components of the CT perfusion data determination facility according to an embodiment of the invention can be embodied mainly in the form of software components. This applies in particular to the image determination unit and the concentration determination unit.

In principle, these components can however also be implemented to some extent, particularly if especially rapid calculations are involved, in the form of software-assisted hardware, for example FPGAs or the like. Similarly, the required interfaces can be embodied, for example where only an acceptance of data from other software components is concerned, as software interfaces. However, they can also be embodied as interfaces constructed from hardware, which are controlled by suitable software.

An implementation largely in software has the advantage that even control facilities of computed tomography systems already in use can be easily upgraded by a software update in order to work in the manner according to an embodiment of the invention. In this respect, an embodiment is directed to a corresponding computer program product with a computer program which is loadable directly into a computed tomography system or a storage facility of a control facility of a computed tomography system and comprises program sections in order to carry out all steps of the CT perfusion data determination method according to an embodiment of the invention and/or the CT perfusion measurement method according to an embodiment of the invention when the computer program is executed in the control facility of the computed tomography system.

Such a computer program product can comprise, where relevant, in addition to the computer program, further components such as for example documentation and/or additional components, including hardware components such as for example hardware keys (dongles, etc.) in order to use the software.

Via a software implementation, the method is executable reproducibly and in a less fault-prone manner on different computers.

For transport to the storage facility of the control facility of the computed tomography system and/or for storage at the control facility of the computed tomography system, a computer-readable medium such as a memory stick, a hard disk or another transportable or firmly installed data carrier can be used on which the program sections of the computer program which are readable and executable by a computer unit of the computed tomography system are stored. For this purpose, the computer unit can have one or more cooperating microprocessors or the like, for example.

The claims and the description below each contain particularly advantageous embodiments and developments of the invention. In particular the claims of one claim category can also be developed similarly to the dependent claims of another claim category. In addition, in the context of the invention, the different features of different example embodiments and claims can also be combined to form new example embodiments. In particular, the features and advantages described in connection with the method according to an embodiment of the invention can also be embodied as corresponding sub-units or modules of the determination facility according to an embodiment of the invention or of the computer program product according to an embodiment of the invention. Conversely, the features and advantages described in connection with the determination facility according to an embodiment of the invention or the computer program product according to an embodiment of the invention can also be embodied as corresponding method steps of the method according to an embodiment of the invention.

According to one embodiment of the CT perfusion data determination method according to an embodiment of the invention, the CT image acquisition method is carried out with a spectrally resolving X-ray detector, preferably a photon-counting X-ray detector. In comparison with a dual-energy CT system or a multi-energy CT system, such an X-ray detector has the advantage that it can be used to acquire or record raw X-ray data from an examination region for the different spectra from the same direction. If, on the other hand, there are two differently positioned X-ray sources, then other objects are easily recorded from the different directions or an examination region is recorded from different perspectives. Furthermore, the X-ray attenuation, in other words the enhancement of the different acquisitions, is also slightly different. These problems are advantageously overcome with the aid of a spectrally resolving X-ray detector, preferably a photon-counting X-ray detector, as in this case the same source is used for different images with different X-ray spectra.

An X-ray tube with a tube voltage of 120 kV and low tube current is preferably operated for CT image acquisition. The acquisition with an X-ray tube voltage of 120 kV makes it possible, especially in the case of iodine as contrast agent, to robustly calculate the iodine-water decomposition, in other words the base material decomposition. With the conventionally preferred 70 kV or 80 kV, on the other hand, the X-ray spectrum is no longer sufficiently broad for such a separation of spectral components into a contrast agent, preferably iodine, and water. The low tube current is attributable simply to the fact that the patient is not permitted to receive too much radiation or should not be exposed to an excessively high radiation dose, which would be the case for a higher tube current. This is because such a perfusion acquisition has a very long duration of 40 to 70 seconds. Furthermore, the reduced image quality due to a reduced tube current is sufficient to produce the native images which according to an embodiment of the invention are now recorded or generated at the same time as the perfusion measurement, as during a perfusion measurement the knowledge of larger regions with an accumulation of contrast agent and the distribution thereof is adequate. Typical values for the by tube currents in conventional CT imaging methods with a tube voltage of 120 kV are exposures of approximately 320 mAs. In the method according to an embodiment of the invention, significantly weaker tube currents can advantageously be used. The exposures corresponding to these weaker tube currents preferably have values of less than 200 mAs, more preferably values of 100 mAs, particularly preferably values of approximately 40 mA, which is associated with significantly lower radiation exposure than in the prior art.

In the CT perfusion data determination method according to an embodiment of the invention, virtual native image data is preferably determined for a plurality of the plurality of perfusion images and thus a plurality of virtual native images are generated for different time instants of the perfusion measurement. In this variant, the baseline image is determined based upon the plurality of virtual native images. Statistically occurring errors in the determination of the baseline image are advantageously reduced, as the data basis for the baseline image is enlarged in comparison to an approach in which only a single virtual native image is used as the basis for a calculation of a baseline image. An increased accuracy and reliability of the baseline image is therefore achieved.

The baseline image is particularly preferably determined based upon the plurality of virtual native images. An averaging enables a reduction of statistical errors in the acquisition of raw X-ray data for the virtual native images.

Moreover, the averaged baseline image forms a particularly favorable average contrast value for the image elements of the native image, so that contrast variations due to temporal changes in the examination region or the image acquisition parameters during the acquisition or generation of the images or image datasets generated in temporal succession for the perfusion measurement or errors caused thereby during the contrast determination of the perfusion images are reduced or minimized.

It is also preferred if, during the CT perfusion measurement according to an embodiment of the invention, a CT perfusion measurement takes place in a first organ of a patient and a concentration of a contrast agent is monitored in another region disposed outside of the examination region, and a start of the perfusion method is triggered when the contrast agent is detected in the region of the region disposed outside of the examination region. Advantageously, a start of the perfusion measurement proper can be synchronized exactly with the arrival of a contrast agent bolus in the examination region. This process is possible because, on account of the fact that additional preliminary acquisitions are dispensed with, a time span of a movement of a contrast agent bolus between the region disposed outside of the examination region and the examination region is sufficient to start the CT perfusion measurement proper.

This method is particularly advantageous if, during the CT perfusion measurement according to an embodiment of the invention, a CT perfusion measurement takes place in the brain of a patient and a concentration of a contrast agent is monitored in another region disposed outside of the examination region, for example an organ, preferably in the heart region of the patient, and a start of the perfusion method is triggered when the contrast agent is detected in the region of the region disposed outside of the examination region, preferably the patient's heart. Here, too, a start of the perfusion measurement proper can advantageously be synchronized exactly with the arrival of a contrast agent bolus in the examination region. As already mentioned, this process is possible because, on account of the fact that additional preliminary acquisitions are dispensed with, a time span of a movement of a contrast agent bolus between the region disposed outside of the examination region, for example the heart region, and the examination region, in particular the brain, is sufficient to start the CT perfusion measurement proper.

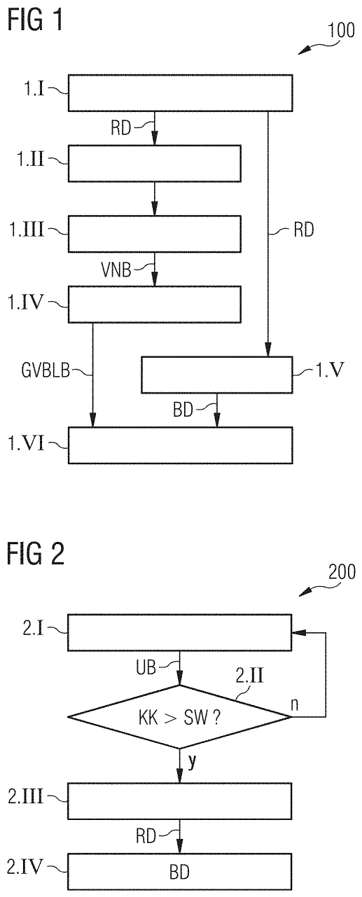

FIG. 1 shows a flow diagram 100 illustrating a CT perfusion data determination method according to an example embodiment of the invention.

In step 1.I, raw data RD affected by contrast agent, which has been detected in a spectrally resolved manner in a CT image acquisition method, is received from an examination region of a patient. In the example embodiment illustrated in FIG. 1, the examination region is a region of the brain. The raw data RD is spectrally resolved and is therefore suitable for a material decomposition. The raw data RD comprises 50 raw datasets, which are assigned to 50 images or image datasets recorded in temporal succession from the examination region.

In step 1.II, a material decomposition into the materials contrast agent and water is performed based upon the raw data RD received. Alternatively, the material decomposition can also take place in the image data space.

Subsequently, in step 1.III a virtual native image VNB is generated based upon the proportion of water of the material decomposition.

Alternatively, the material proportions could also be combined to form mono-energy image data VNB. Here, the material proportions of the X-ray attenuation in either the raw data space or the image data space are combined in such a way that image data with a predetermined high X-ray energy is generated. The virtual image data VNB is then calculated for a high predetermined X-ray energy above the X-ray edge. In other words, the X-ray energy is selected in such a way that a native image is produced, in other words without consideration of the properties of the contrast agent for the selected energy. In the example embodiment illustrated in FIG. 1, a native image VNB is calculated for each of the 50 received images.

In step 1.IV, an averaged virtual baseline image GVBLB is calculated by averaging the 50 native images VNB calculated in step 1.III. The averaged baseline image forms a particularly favorable average contrast value for the image elements of the native image, so that contrast variations due to temporal changes in the examination region or the image acquisition parameters during the acquisition or generation of the 50 image datasets generated in temporal succession are reduced or minimized.

Moreover, in step 1.V 50 images BD affected by contrast agent are also reconstructed based upon the raw data RD.

Finally, in step 1.VI a course of a contrast agent concentration, in other words a spatially and temporally variable contrast agent concentration in the examination region is determined based upon the 50 image datasets BD affected by contrast agent and based upon the averaged virtual baseline image GVBLB. Here the averaged virtual baseline image GVBLB is subtracted from the image datasets BD affected by contrast agent in order to determine the X-ray attenuation through the contrast agent alone, for example iodine, in the examination region.

FIG. 2 shows a flow diagram 200 illustrating a CT perfusion method according to an example embodiment of the invention. Firstly, step 2.I involves carrying out a CT image acquisition UB or several such CT image acquisitions in temporal succession of a patient's heart. Based on the CT image acquisition UB or the image data generated thereby, it is determined in step 2.II whether a contrast agent concentration KK has exceeded a predetermined value SW in the region of the heart. Such a monitoring of the contrast agent concentration can take place for example by way of a plurality of CT acquisitions carried out in temporal succession. In addition to the heart region, a small section of the aorta or a section of the carotid artery are also suitable for this purpose.

If it has been determined in step 2.II that the contrast agent K has arrived at the heart, which is indicated in FIG. 2 with “y”, the method proceeds to step 2.III. If it has been determined in step 2.II that the contrast agent K has not yet arrived at the patient's heart, which is indicated in FIG. 2 with “n”, the method reverts to step 2.I.

In step 2.III, the perfusion measurement proper is now started since, based on the knowledge that the contrast agent has arrived in the heart region, a time of arrival of the contrast agent in the brain can be determined or estimated and thus the start time of the perfusion measurement in the brain established. Furthermore, during this perfusion measurement a spectral CT image acquisition method is carried out on the patient's brain, wherein 50 raw X-ray datasets RD affected by contrast agent are generated or 50 image acquisitions are carried out at time instants t1, . . . , t50.

In step 2.IV, the CT perfusion data determination method illustrated in FIG. 1 is carried out based upon the raw X-ray datasets RD obtained, wherein 50 contrast agent images BD are determined, which show the temporal course of a contrast agent concentration or a spatially and temporally variable distribution of the contrast agent in the patient's brain.

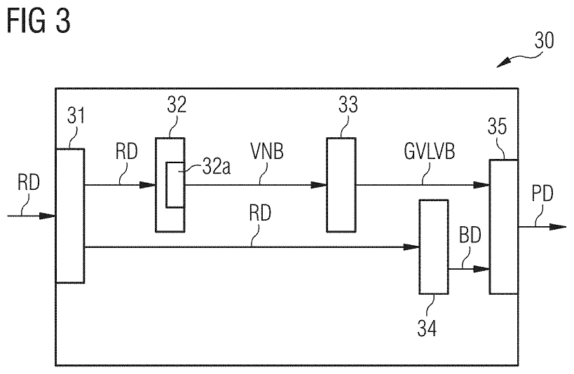

FIG. 3 shows a schematic representation of a CT perfusion data determination facility 30 according to an example embodiment of the invention.

The CT perfusion data determination facility 30 comprises an X-ray data acquisition unit 31. The X-ray data acquisition unit 31 receives 50 raw datasets RD for a total of 50 images acquired in the presence of contrast medium, which have been recorded by an X-ray detector during a CT image acquisition from an examination region of a patient.

The CT perfusion data determination facility 30 also comprises an image determination unit 32 for determining virtual baseline images based upon the recorded raw X-ray datasets RD.

A material decomposition unit 32a is also part of the image determination unit 32. The material decomposition unit 32a generates virtual native images VNB based upon a material decomposition of the recorded raw X-ray data, for example for a plurality, preferably for each of the recorded raw X-ray datasets RD in order to generate virtual baseline images.

The CT perfusion data determination facility 30 also comprises an averaging unit 33, which is configured to calculate an averaged virtual baseline image GVBLB by averaging the generated virtual native image datasets VNB or virtual baseline images based thereon.

The CT perfusion data determination facility 30 further comprises an image reconstruction unit 34, which is configured to reconstruct image datasets BD based upon the recorded raw X-ray datasets RD, which are the basis for a perfusion measurement.

The averaged virtual baseline image GVBLB is transmitted to a concentration determination unit 35, which is also part of the CT perfusion data determination facility 30. The concentration determination unit 35 is configured to determine a temporal course of a contrast agent concentration K, in other words a spatially dependent and time-dependent contrast agent concentration in the examination region based upon the reconstructed image datasets BD and the averaged virtual baseline image GVBLB. The determined perfusion measurement data PD is then output for display or further processing, for example.

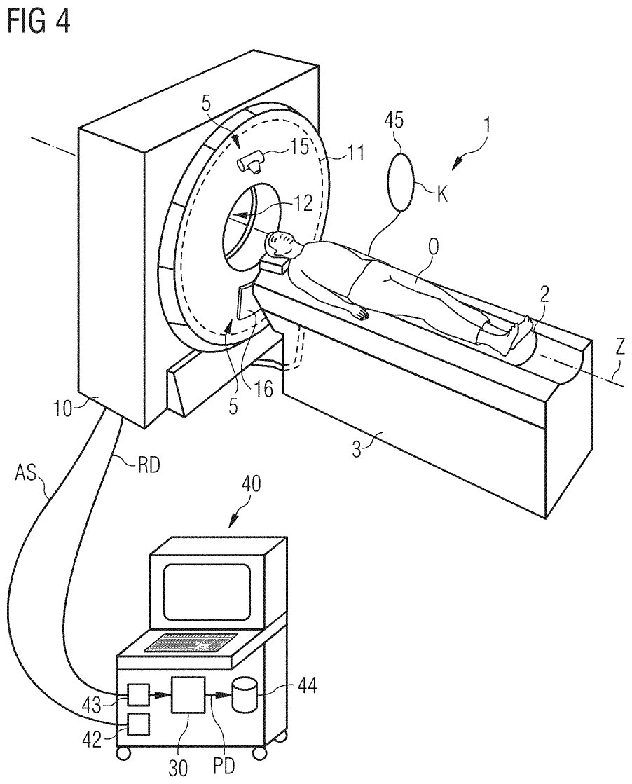

FIG. 4 illustrates a CT system 1 according to an example embodiment of the invention.

The CT system 1, which is embodied as a CT system with a photon-counting detector 16, consists primarily of a standard scanner 10, in which, on a gantry 11, a projection measurement data acquisition unit 5 with a photon-counting detector 16 and an X-ray source 15, which is located opposite the photon-counting detector 16, rotates about a measurement space 12. Situated in front of the scanner 10 is a patient positioning facility 3 or patient table 3, the upper part 2 of which can be displaced relative to the scanner 10 with a patient O situated thereon in order to move the patient O through the measurement space 12 relative to the detector system or the detector 16. The scanner 10 and the patient table 3 are controlled by a control facility 40 from which acquisition control signals AS come via a conventional control interface 42 in order to control the entire system in the conventional manner according to predetermined measurement protocols. In the case of spiral acquisition, movement of the patient O along the z-direction, which corresponds to the system axis z lengthwise through the measurement space 12, and the simultaneous rotation of the X-ray source 15 result in a helical path for the X-ray source 15 relative to the patient O during the measurement. The detector 16 runs in parallel with this process always opposite the X-ray source 15 in order to record spectrally resolved projection measurement data RD, which is then used to reconstruct volume and/or slice image data. It is equally possible to carry out a sequential measurement method in which travel is performed to a fixed position in the z-direction, and then during one revolution, part of a revolution or a plurality of revolutions at the z-position concerned, the required spectrally resolved projection measurement data RD is recorded in order to reconstruct a sectional image at this z-position or to reconstruct image data from the projection measurement data at a plurality of z-positions. In principle, the CT perfusion determination method according to an embodiment of the invention and the perfusion measurement method according to an embodiment of the invention can also be applied to or carried out on other CT systems, for example comprising a plurality of X-ray sources or comprising a full-ring detector. For example, the methods according to an embodiment of the invention can also be applied to a system comprising an immobile patient table and a gantry that moves in the z-direction (known as a sliding gantry).

The spectrally resolved projection measurement data RD (also referred to below as raw data) acquired by the detector 16 is passed to the control facility 40 via a raw-data interface 43. This raw data RD is then processed, if applicable after suitable preprocessing, in a CT perfusion data determination facility 30, which in this example embodiment is implemented in the form of software on a processor in the control facility 40. The perfusion data determination facility 30 is set up as illustrated in FIG. 3 and generates perfusion measurement data PD from the recorded raw data RD.

The perfusion measurement data PD generated by the perfusion data determination facility 30 is then stored in a memory 44 of the control facility 40 and/or output in the usual manner on the screen of the control facility 40. It can also be supplied via an interface (not shown in FIG. 4) to a network connected to the computed tomography system 1, for example to a radiology information system (RIS), and stored in a mass storage facility accessible there, or output as images on printers or filming stations connected there. The data can thus be further processed in any desired manner and then stored or output.

FIG. 4 also shows a contrast agent injection facility 45, which is used to inject the patient O with a contrast agent K in preparation for a perfusion measurement, in other words before the CT perfusion measurement method starts. The regions through which the contrast agent K flows can then be recorded as images during the perfusion measurement with the aid of the computed tomography system 1.

The components of the perfusion data determination facility 30 can be implemented mostly or entirely in the form of software elements on a suitable processor. In particular, the interfaces between these components can also be embodied purely in the form of software. The only requirement is that suitable memory regions are accessible in which the data is stored temporarily in a suitable manner and can be retrieved and updated at any time.

Finally, it should again be noted that the methods and facilities described above are merely preferred example embodiments of the invention and that the invention can also be varied by a person skilled in the art without departing from the scope of the invention as defined by the claims. For the sake of completeness, it should be noted that the use of the indefinite articles “a” or “an” does not preclude the relevant features from also being present plurally. Similarly, the expression “unit” does not preclude this consisting of a plurality of components which can possibly also be spatially distributed.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A CT perfusion data determination method, comprising:
recording raw X-ray data affected by contrast agent and generated by way of spectral or multi-energy CT image acquisition, wherein a plurality of images have been acquired from an examination region at successive time instants;
determining a virtual baseline image by calculating virtual native image data by way of a material decomposition based upon the raw X-ray data recorded; and
determining a temporal course of a contrast agent concentration in the examination region based upon the raw X-ray data affected by contrast agent and the virtual baseline image.

2. The method of claim 1, wherein the CT image acquisition method is carried out with a photon-counting X-ray detector.

3. The method of claim 1, wherein an X-ray tube with 120 kV and low tube current is operated for CT image acquisition.

4. The CT method of claim 1, wherein, for each respective image of the plurality of images, a respective virtual native image is determined based upon a material decomposition and the baseline image is determined based upon the plurality of respective virtual native images generated.

5. The method of claim 4, wherein the baseline image is determined as an averaged image based upon the plurality of respective virtual native images.

6. A CT perfusion measurement method, comprising:
carrying out the spectral or multi-energy CT image acquisition, wherein raw X-ray image data affected by contrast agent is generated from an examination region and the plurality of images are acquired from the examination region at successive time instants; and
carrying out the CT perfusion data determination method of claim 1 based upon the raw X-ray data affected by contrast agent recorded.

7. The method of claim 6, wherein
a perfusion measurement takes place in a first body region of a patient;
a concentration of a contrast agent in the first body region is monitored, and
a start of the perfusion measurement method is triggered in a second body region when the contrast agent is detected in the first body region of the patient.

8. A CT perfusion data determination facility, comprising:
a data acquisition unit to record raw X-ray data affected by contrast agent and generated by way of spectral or multi-energy CT image acquisition, wherein a plurality of images have been acquired from the examination region at successive time instants;
an image determination unit to determine a virtual baseline image by calculating virtual native image data by way of a material decomposition based upon the raw X-ray data recorded; and
a concentration determination unit to determine a temporal course of a contrast agent concentration in the examination region based upon the raw X-ray data affected by contrast agent and the virtual baseline image.

9. A computed tomography system, comprising:
an X-ray emission unit;
a detector unit to record spectral or multi-energy raw data; and
the CT perfusion data determination facility of claim 8.

10. A non-transitory computer program product storing a computer program, directly loadable into a storage facility of a computed tomography system, including program sections to carry out the method of claim 1 when the computer program is executed in the computed tomography system.

11. A non-transitory computer-readable medium storing program sections, readable in and executable by a computer unit, to carry out the method of claim 1 when the program sections are executed by the computer unit.

12. The method of claim 2, wherein an X-ray tube with 120 kV and low tube current is operated for CT image acquisition.

13. The CT method of claim 2, wherein, for each respective image of the plurality of images, a respective virtual native image is determined based upon a material decomposition and the baseline image is determined based upon the plurality of respective virtual native images generated.

14. The method of claim 13, wherein the baseline image is determined as an averaged image based upon the plurality of respective virtual native images.

15. The method of claim 7, wherein the first body region is a heart of the patient and the second body region is the brain of the patient.

16. A non-transitory computer program product storing a computer program, directly loadable into a storage facility of a computed tomography system, including program sections to carry out the method of claim 2 when the computer program is executed in the computed tomography system.

17. A non-transitory computer-readable medium storing program sections, readable in and executable by a computer unit, to carry out the method of claim 2 when the program sections are executed by the computer unit.
